(19) United States
(12) Patent Application Publication
(10) Pub. No.: US 2010/0292620 A1
(43) Pub. Date: Nov. 18, 2010

(54) Design a cast

(76) Inventor: Connie Lattimore, Matteson, IL (US)

Correspondence Address: RUTH EURE, 4795 EDISON AVENUE, BOULDER, CO, 80301, US

(21) Appl. No.: 12/800,226

(22) Filed: May 10, 2010

Related U.S. Application Data
(60) Provisional application No. 61/216,294, filed on May 15, 2009.

(51) Int. Cl. A61F 5/01

(52) U.S. Cl. 602/3

(57) ABSTRACT

This invention provides a line of decorative fabric sheathes to be easily applied over any hand, wrist, arm, foot or leg cast, providing the wearer a dramatic means of enhancing the appearance of the cast and cleverly hiding it from view. Produced from a variety of materials including sturdy cotton, fancy lace and soft suede to name a few options, the Design A Cast functions is a fitted sleeve, providing a snug, yet comfortable fit around the wearer's affected limb. As such, the Design A Cast is generally tubular in shape. The present invention can be rolled in a tight coil between uses and unrolled as it is pulled up and over the actual cast.

### Claim of Priority

This patent application claims priority under 35 USC 119 (e) (1) from U.S. Provisional Patent Application Ser. No. 61/216,294filed May 15, 2010, of common inventorship herewith entitled, “Design A Cast.”

### FIELD OF THE INVENTION

The present invention pertains to the field of decoration for plaster or other immobilization casts.

### BACKGROUND OF THE INVENTION

The prior art has put forth several designs for the decoration or covering of plaster casts. Among these are:

U.S. Pat. No. 6,126,621 to Aceves describes a cast cover which comprises a cloth cover which slides over the cast in a loose fitting manner having a cuff assembly at an open end, and a plurality of cinch assemblies.

U.S. Pat. No. 7,066,900 to Baron describes a cast cover and method of use for a hand, forearm or wrist cast comprising a tubular portion for covering the cast, a distal portion including a thumb hole for receiving a thumb of the user and an oval finger opening for receiving the fingers of the user, and finger separators spanning the oval finger opening.

U.S. Pat. No. D484,604 describes a design for a decorative cast cover having smiling faces imprinted thereon.

None of these references describes the present invention.

### SUMMARY OF THE INVENTION

It is an object of the present invention to provide a decorative sheath to be easily applied over any wrist, arm, leg, foot, ankle, neck, finger, knee or any other broken limb which would require a cast.

### BRIEF DESCRIPTION OF THE DRAWINGS

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 is a picture depicting the present invention on an adult arm sleeve cast showing a dragon.

FIG. 2 is a picture depicting the an alternative embodiment of the present invention on an arm sleeve cast showing rhinestones.

FIG. 3 is a picture depicting the present invention on an adult leg sleeve cast showing closed toe.

FIG. 4 is a picture depicting the present invention on an adult leg sleeve cast showing open toe.

FIG. 5 is a picture depicting the present invention on a children's arm sleeve cast showing Halloween pumpkins.

FIG. 6 is a picture depicting the present invention on a children's arm sleeve cast showing baby rattles.

FIG. 7 is a picture depicting the present invention on a toddler's leg sleeve cast showing closed toe decorated with racecars.

FIG. 8 is a picture depicting the present invention on a toddler's leg sleeve cast showing open toe decorated with racecars.

FIG. 9 is a picture depicting the present invention on a dog's leg sleeve cast showing dog bones.

FIG. 10 is a picture depicting the present invention on a cat's leg sleeve cast showing paw prints.

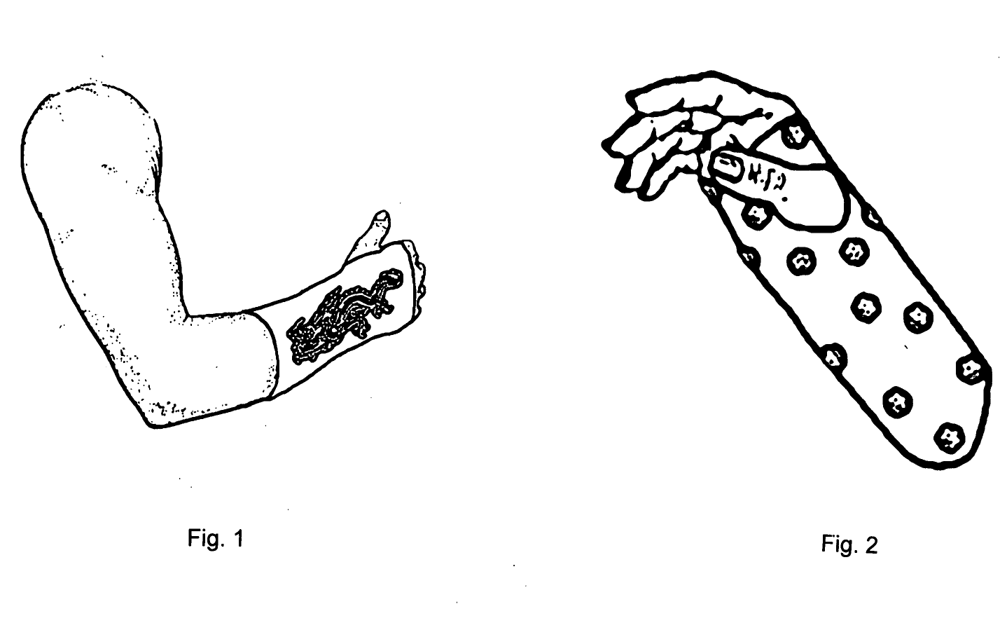

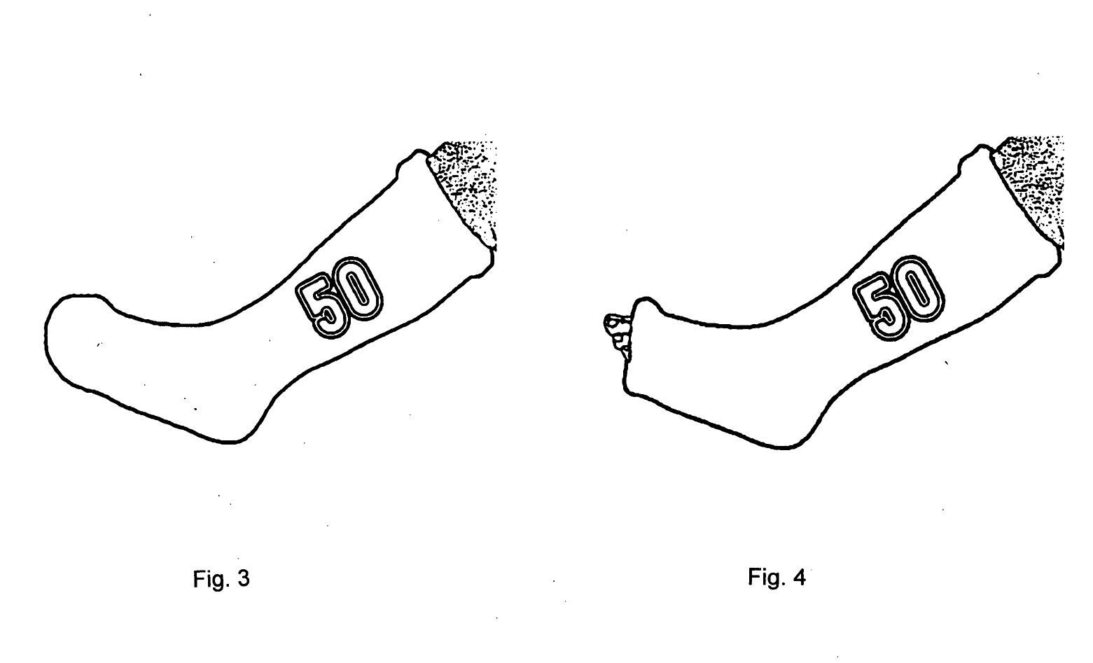

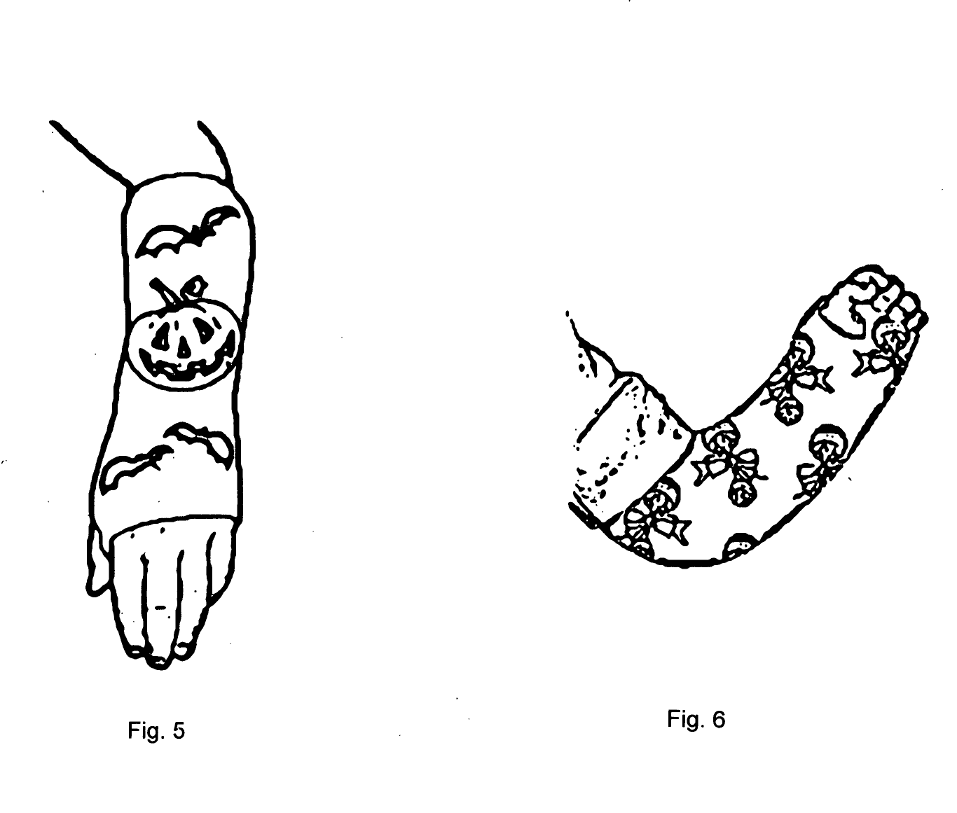

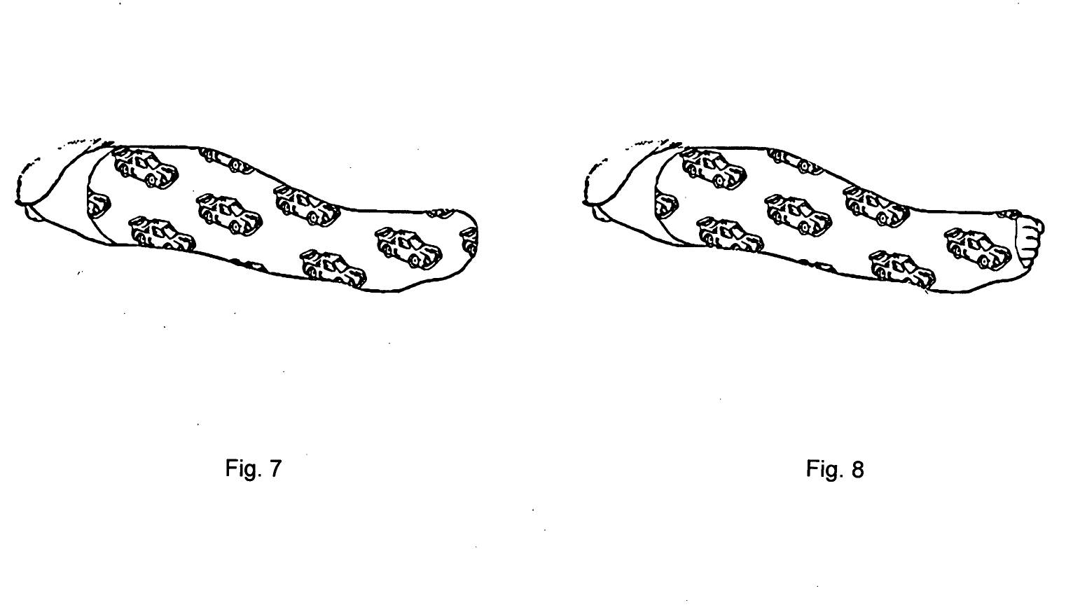

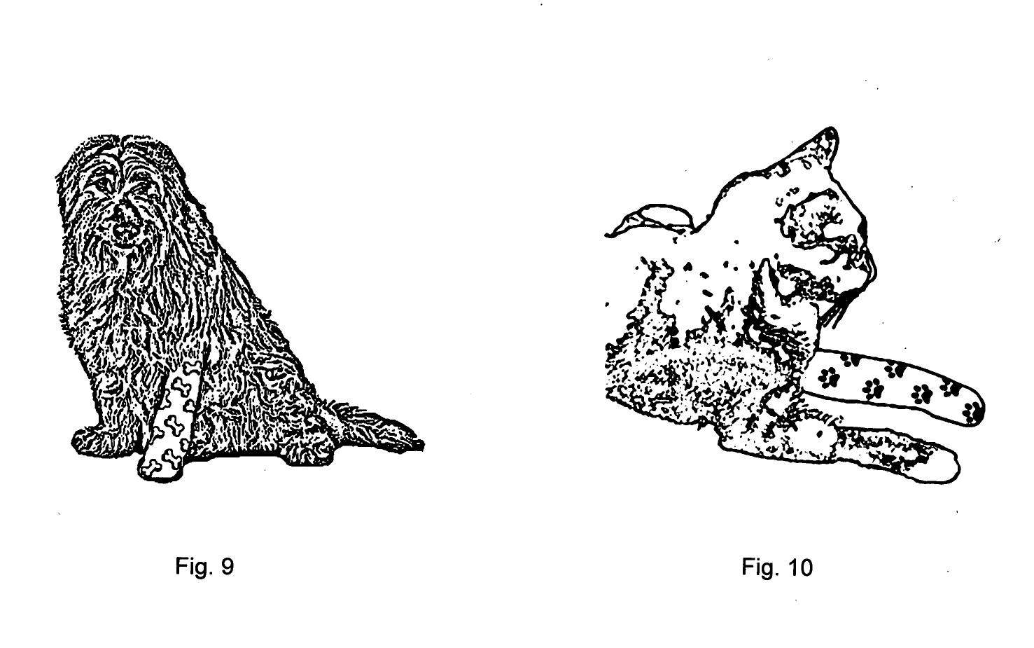

### DETAILED DESCRIPTION OF THE INVENTION

Every year, thousands of Americans suffer from broken bones. Whether twisting an ankle after tripping down a flight of stairs, fracturing a leg in an unfortunate skiing accident or falling off a scooter and breaking one's arm, fractures are a painful injury that are typically slow to heal. The most common treatment for a fracture is setting the broken bones into proper alignment, then wrapping the injured area in a plaster or resin-coated cast. The purpose of a cast is to hold the broken bones in place as they heal, restricting movement and ensuring that it heals properly, a process that can take anywhere from a few days to several weeks, depending upon the severity of the fracture.

While wearing a cast is an important part of the healing process, they can be a nuisance. A constant reminder of a painful injury, the very presence of a cast can be depressing, reminding the user of activities and events that their injury is preventing them from actively pursuing. Further, a typical cast tends to be very bland in appearance. Casts are most often manufactured of plain white or colored plaster material. Easily sullied by exposure to dirt and debris, the surface of a cast can quickly become dull and dirty, creating quite an eyesore. Not surprisingly, many style conscious consumers are unhappy to find that a dull cast can detract from a carefully planned ensemble.

Having recognized the potential for a fun and novel means of “dressing up” a cast, the present invention was developed and is hereinafter referred to the Design A Cast. This invention provides a line of decorative fabric sheathes to be easily applied over any hand, wrist, arm, foot or leg cast, providing the wearer a dramatic means of enhancing the appearance of the cast and cleverly hiding it from view. Produced from a variety of materials including sturdy cotton, fancy lace and soft suede to name a few options, the Design A Cast functions is a fitted sleeve, providing a snug, yet comfortable fit around the wearer's affected limb. As such, the Design A Cast is generally tubular in shape. The present invention can be rolled in a tight coil between uses and unrolled as it is pulled up and over the actual cast.

In an embodiment, the Design A Cast comprises an elastic threading, as well as flexible, elastic banding at the openings, providing a snug, custom like fit. The Design A Cast is produced in a variety of sizes appropriate for arm, foot or leg casts, with sizes appropriate for both children, adults and animals made available.

Most notably, this product can be manufactured in a myriad of fun and funky colors, as well as in novelty prints. For instance, Design A Cast sheathes boasting floral patterns, camouflage prints and fancy rhinestone or beaded accents could all be made available. Further, the Design A Cast could be adorned with the logos of various sports teams, as well as corporate insignia. This product line could also be adorned with favorite cartoon characters and the images of pop culture icon. As can be imagined, design considerations for this decorative cast cover are virtually limitless. It is important to note that in the case of coverings adorned with logos, insignia or other licensed images, licensing concerns must be addressed.

Use of the Design A Cast is very straight forward and simple. First, the consumer chooses the size Design A Cast appropriate for their injury. By way of example, a person suffering from a broken wrist would select a small sized Design A Cast, while a person with a broken leg would select a larger version. The user then purchases the Design A Cast according to the desired material and design. For instance, a teenage girl might employ a fancy pink lace Design A Cast, embellished with silk ribbons and rhinestones to wear over a wrist cast, while a diehard fan of the Chicago Bears might utilize a navy blue Design A Cast adorned with the Bears logo, applying the unit over a foot cast. Next, the user simply dons the Design A Cast according to easily followed package instructions. Placing the rolled Design A Cast at the base of their cast, the user simply slides the Design A Cast up and over the cast, unrolling the unit as they go. With the Design A Cast properly donned, the user simply goes about their scheduled activities, removing and laundering the covering as needed.

There are many significant benefits and advantages associated with the Design A Cast. Foremost, this cleverly designed invention enables those suffering from a broken limb to customize their cast to suit their own personal interests and sense of style. An entire line of decorative sleeves produced in myriad of attractive colors, patterns and printed designs, the Design A Cast turns a mundane arm or leg cast into a fancy showpiece. Style conscious persons will especially appreciate the Design A Cast as they could pick and choose various coverings to coordinate with specific ensembles. Adding a fancy decorative touch to a medical appliance, the Design A Cast also appeals to children and young adults. Providing an attractive means of camouflaging a sullied or bland cast, the Design A Cast serves to lighten spirits and alleviate the trauma associated with a broken limb.

Another advantage is that the Design A Cast is convenient and efficient to use. Easily applied simply by sliding the unit up and over the injured limb, the Design A Cast can be donned and removed in a manner of seconds. Machine washable, this decorative covering will withstand repeated use, with ease.

Innovative and unique, the Design A Cast is a practical invention which offers those with a broken limb a simple means of adding a splash of color and flair to their cast. Striking in design and easy to use, this unique invention will prove to be a favorite commodity of any style conscious consumer.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A covering for a cast, comprising any suitable material, including sturdy cotton, fancy lace and soft suede, in the form of a generally tubular fitted sleeve, providing a snug, yet comfortable fit around a wearer's affected limb, wherein the tube can be rolled into a tight coil between uses and unrolled as it is pulled up and over the actual cast.

2. The covering for a cast of claim 1 comprising an elastic threading ,and flexible, elastic banding at the openings, providing a snug, custom like fit.

3. The covering for a cast of claim produced in a variety of sizes appropriate for arm, foot or leg casts, with sizes appropriate for children, adults and animals.

4. A method of decorating and protecting a cast, comprising:
choosing the tube size appropriate for their injury, comprising desired material and design;
placing the rolled tube at the base of the cast;
sliding the tube up and over the cast, unrolling the tube as the cast is covered;
removing and laundering the tube as needed.
